SEQUENCE LISTING (13 sequences)

SEQ ID NO: 1 - DNA, 20 bp, synthetic construct
  source 1..20: mol_type = other DNA
gacctgccgtctagaaaaac

SEQ ID NO: 2 - DNA, 20 bp, synthetic construct
  source 1..20: mol_type = other DNA
ctgtagccaaattcgttgtc

SEQ ID NO: 3 - DNA, 20 bp, synthetic construct
  source 1..20: mol_type = other DNA
acctatgcctggcacacttc

SEQ ID NO: 4 - DNA, 20 bp, synthetic construct
  source 1..20: mol_type = other DNA
aggaagtagcctcccacgat

SEQ ID NO: 5 - DNA, 20 bp, synthetic construct
  source 1..20: mol_type = other DNA
tgggatcattgccctgtgag

SEQ ID NO: 6 - DNA, 20 bp, synthetic construct
  source 1..20: mol_type = other DNA
ggtgtctgaaggagggggta

SEQ ID NO: 7 - DNA, 20 bp, synthetic construct
  source 1..20: mol_type = other DNA
ccccagtcacctgctgttat

SEQ ID NO: 8 - DNA, 20 bp, synthetic construct
  source 1..20: mol_type = other DNA
agatctccttggccacaatg

SEQ ID NO: 9 - DNA, 21 bp, synthetic construct
  source 1..21: mol_type = other DNA
ttttggtgttgtgcaagggtc

SEQ ID NO: 10 - DNA, 20 bp, synthetic construct
  source 1..20: mol_type = other DNA
atcgctccctctccctgtaa

SEQ ID NO: 11 - DNA, 21 bp, synthetic construct
  source 1..21: mol_type = other DNA
atcgctaccctgctgttattc

SEQ ID NO: 12 - DNA, 21 bp, synthetic construct
  source 1..21: mol_type = other DNA
acgtcaatgcgtgcttctttg

SEQ ID NO: 13 - DNA, 21 bp, synthetic construct
  source 1..21: mol_type = other DNA
tcaacctgtttgccatgtttg

(19) United States
(12) Patent Application Publication
(10) Pub. No.: US 2024/0058311 A1
(43) Pub. Date: Feb. 22, 2024

(54) USE OF SMO INHIBITOR IN PREPARATION OF DRUG FOR PREVENTING, DELAYING OR ALLEVIATING ACCESS STENOSIS OF ARTERIOVENOUS FISTULA

(71) Applicant: JINAN UNIVERSITY, Guangzhou, Guangdong (CN)

(72) Inventors: Hui ZENG, Guangzhou (CN); Juan DU, Guangzhou (CN); Heng WAN, Guangzhou (CN); Heng MENG, Guangzhou (CN); Kexiu HUANG, Guangzhou (CN)

(21) Appl. No.: 18/355,219

(22) Filed: Jul. 19, 2023

(30) Foreign Application Priority Data
Jul. 20, 2022 (CN) 202210863481.3

(51) Int. Cl. A61K 31/4355; A61K 31/4418; A61K 31/454; A61P 9/14

(52) U.S. Cl. CPC A61K 31/4355; A61K 31/4418; A61K 31/454; A61P 9/14

(57) ABSTRACT

Provided is use of a SMO inhibitor in the preparation of a drug for preventing, delaying or alleviating access stenosis of arteriovenous fistula. It is creatively found herein that a SMO inhibitor can protect endothelial cells under the stimulation of high glucose and alleviate endothelial cell dysfunction, and specifically reduce lactate dehydrogenase (LDH), reduce apoptosis and reduce the production of inflammatory cytokines TNF-α, MCP-1, and IL-6. Furthermore, it is found herein that the SMO inhibitor can improve endothelial cell dysfunction by increasing the blood flow velocity in blood vessels, increasing the inner diameter of the blood vessel, improving the vascular intima thickening and reducing the production of inflammatory cytokines, thereby achieving the purpose of preventing, delaying or alleviating the access stenosis of arteriovenous fistula.

### CROSS REFERENCE TO RELATED APPLICATION(S)

This application claims the benefit of Chinese Application No. 202210863481.3, entitled “USE OF SMO INHIBITOR IN PREPARATION OF DRUG FOR PREVENTING, DELAYING OR ALLEVIATING ACCESS STENOSIS OF ARTERIOVENOUS FISTULA” and filed on Jul. 20, 2022, the disclosure of which is expressly incorporated by reference herein in its entirety.

### REFERENCE TO ELECTRONIC SEQUENCE LISTING

The application contains a Sequence Listing which has been submitted electronically in .XML format and is hereby incorporated by reference in its entirety. Said .XML file, created on Jun. 15, 2023, is named “038480.00135 Sequence Listing.xml” and is 65,536 bytes in size. The sequence listing contained in this .XML file is part of the specification and is hereby incorporated by reference herein in its entirety.

### TECHNICAL FIELD

The present disclosure belongs to the medical field and relates to use of a smoothened (SMO) inhibitor in the preparation of a drug for preventing, delaying or alleviating access stenosis of arteriovenous fistula.

### BACKGROUND

Diabetes mellitus (DM) is a major public health problem worldwide and an established independent risk factor for cardiovascular events and cardiovascular death. Diabetes is typically characterized by high blood glucose. The most common type of diabetes is type 1 diabetes, and in such diabetes, the absolute lack of insulin leads to the destruction of pancreatic cells. In addition, there is type 2 diabetes, in which insulin resistance can lead to high blood glucose. Diabetes is prone to various complications, and these complications involve almost every tissue of the body. Meanwhile, diabetes is the main cause of high cardiovascular morbidity and high mortality, blindness, renal failure, and amputation. In addition, diagnosis of type 2 diabetes early in adolescents and young people under the age of 40 is related to the severity of the disease, which can lead to the premature development of serious complications.

Diabetic nephropathy (DN) is a common complication of diabetes and the main cause of chronic renal diseases. About 40% of diabetic patients develop diabetic nephropathy which is characterized by proteinuria, increased blood pressure, and reduced renal function, and develop end-stage renal disease (ESRD). These thought-provoking statistics underscore the importance of tracing the root causes of diabetes and complications thereof to provide the best intervention treatment for such a disease. When diabetic nephropathy develops into ESRD, dialysis is required to improve the condition.

Arteriovenous fistula (AVF) is the first choice of vascular access for hemodialysis in patients with end-stage renal diseases. AVF has long service life and fewer complications and creates favorable conditions for hemodialysis treatment. However, the primary patency rate of AVF is low. A meta-analysis shows that the one-year primary patency rate of AVF is 60% and the two-year primary patency rate is 51%. Percutaneous transluminal angioplasty (PTA) is the first-line treatment for AVF dysfunction caused by vascular stenosis. However, some patients will face secondary stenosis after recanalization of the hemodialysis access. After PTA, restenosis caused by venous neointimal hyperplasia (VNH) leads to restenosis of AVE resulting in poor patency. Studies have shown that the one-year secondary patency rate is 71% and two-year secondary patency rate is 64%.

With the development of the economy and the extension of life expectancy, the prevalence of diabetes mellitus is increasing rapidly. Diabetes and complications thereof threaten the health and life of patients, even lead to disability and premature death, and cause a huge waste of funds and resources to society. Diabetes damages large and small blood vessels of the whole body. Therefore, places where blood vessels are concentrated become the “hardest-hit areas” of diabetic complications, including kidneys, large and medium blood vessels, retina, nervous system, and so on. The annual medical expenses are huge. The prevention and treatment of diabetes and complications thereof is a major public health problem faced by the present disclosure.

In summary, although AVF for diabetic nephropathy is a treatment scheme and good news for most patients, some patients will suffer from fistula stenosis after frequent dialysis, and the patency is low. Even with surgical interventions for recanalization after stenosis, the secondary patency is not ideal. The mechanism of fistula stenosis is involved herein, but at present, the specific mechanism is not clear enough, and there is still a lack of effective prevention and treatment methods and follow-up treatment targets. Therefore, the present disclosure urgently needs to find new therapeutic targets and safe and effective new drugs to relieve and alleviate the key molecular events in the pathogenetic process of AVF stenosis in diabetic nephropathy.

### SUMMARY

In view of the deficiency in the existing art, the present disclosure is to provide use of a SMO inhibitor in the preparation of a drug for preventing, delaying or alleviating access stenosis of arteriovenous fistula. The access stenosis of arteriovenous fistula includes arteriovenous fistula stenosis in hemodialysis of diabetic nephropathy.

In a first aspect, the present disclosure provides use of a SMO inhibitor in the preparation of a drug for preventing, delaying or alleviating access stenosis of arteriovenous fistula.

Preferably, the SMO inhibitor includes any one or a combination of at least two of cyclopamine, vismodegib or glasdegib. The combination of at least two of the preceding SMO inhibitors is, for example, a combination of cyclopamine and vismodegib, a combination of glasdegib and cyclopamine, a combination of vismodegib and glasdegib, or a combination of cyclopamine, vismodegib and glasdegib.

Preferably, the SMO inhibitor includes cyclopamine.

Preferably, a dosage form of the drug includes a solution, a tablet, a capsule or a granule.

Preferably, the drug also includes a pharmaceutically acceptable adjuvant.

Preferably, the adjuvant includes any one or a combination of at least two of a diluent, a disintegrant, a flavoring agent, an adhesive, an excipient or a filler.

The present disclosure further provides use of the SMO inhibitor in the preparation of a product for preventing, delaying or alleviating access stenosis of arteriovenous fistula with non-diagnostic/therapeutic purposes. The product is, for example, animal feed additives, and can be used in scientific research related to access stenosis of arteriovenous fistula.

In a second aspect, the present disclosure provides use of a SMO inhibitor in the preparation of a drug for preventing or treating vasculitis caused by endothelial injury.

Preferably, the SMO inhibitor includes any one or a combination of at least two of cyclopamine, vismodegib or glasdegib.

Preferably, the SMO inhibitor includes cyclopamine.

In a third aspect, the present disclosure provides use of a SMO inhibitor in the preparation of a drug for maintaining or improving the blood flow velocity in blood vessels.

The present disclosure further provides use of the SMO inhibitor in the preparation of a product for maintaining or improving the blood flow velocity in blood vessels with non-diagnostic/therapeutic purposes. The product is, for example, animal feed additives, and can be used in scientific research related to blood vessels.

Preferably, the SMO inhibitor includes any one or a combination of at least two of cyclopamine, vismodegib or glasdegib.

Preferably, the SMO inhibitor includes cyclopamine.

In a fourth aspect, the present disclosure provides use of a SMO inhibitor in the preparation of a drug for increasing the inner diameter of the blood vessel.

The present disclosure further provides use of the SMO inhibitor in the preparation of a product for increasing the inner diameter of the blood vessel with non-diagnostic/therapeutic purposes. The product is, for example, animal feed additives, and can be used in scientific research related to blood vessels.

Preferably, the SMO inhibitor includes any one or a combination of at least two of cyclopamine, vismodegib or glasdegib.

Preferably, the SMO inhibitor includes cyclopamine.

In a fifth aspect, the present disclosure provides use of a SMO inhibitor in the preparation of a drug for preventing or improving vascular intima thickening.

The present disclosure further provides use of the SMO inhibitor in the preparation of a product for improving vascular intima thickening with non-diagnostic/therapeutic purposes. The product is, for example, animal feed additives, and can be used in scientific research related to blood vessels.

Preferably, the SMO inhibitor includes any one or a combination of at least two of cyclopamine, vismodegib or glasdegib.

Preferably, the SMO inhibitor includes cyclopamine.

In a sixth aspect, the present disclosure provides use of a SMO inhibitor in the preparation of a drug for reducing lactate dehydrogenase.

The present disclosure further provides use of the SMO inhibitor in the preparation of a product for reducing lactate dehydrogenase with non-diagnostic/therapeutic purposes. The product is, for example, animal feed additives, and can be used in scientific research related to lactate dehydrogenase.

Preferably, the SMO inhibitor includes any one or a combination of at least two of cyclopamine, vismodegib or glasdegib.

Preferably, the SMO inhibitor includes cyclopamine.

In a seventh aspect, the present disclosure provides use of a SMO inhibitor in the preparation of a TNF-α antagonist, an IL-6 antagonist or an MCP-1 antagonist.

The present disclosure further provides use of the SMO inhibitor in the preparation of a TNF-α antagonist, an IL-6 antagonist or an MCP-1 antagonist with non-diagnostic/therapeutic purposes. The use is, for example, the basic research related to the TNF-α antagonist, IL-6 antagonist or MCP-1 antagonist.

Preferably, the SMO inhibitor includes any one or a combination of at least two of cyclopamine, vismodegib or glasdegib.

Preferably, the SMO inhibitor includes cyclopamine.

In an eighth aspect, the present disclosure provides use of a SMO inhibitor in the preparation of an apoptosis inhibitor for vascular endothelial cells.

The present disclosure further provides use of the SMO inhibitor in the preparation of an apoptosis inhibitor for vascular endothelial cells with non-diagnostic/therapeutic purposes. The use is, for example, the basic research related to the apoptosis mechanism of vascular endothelial cells.

Preferably, the SMO inhibitor includes any one or a combination of at least two of cyclopamine, vismodegib or glasdegib.

Preferably, the SMO inhibitor includes cyclopamine.

In a ninth aspect, the present disclosure provides use of a SMO inhibitor in the preparation of food, a health product or a drug for decreasing blood glucose.

The present disclosure further provides use of the SMO inhibitor in the preparation of a hypoglycemic product with non-diagnostic/therapeutic purposes. The product is, for example, animal feed additives, and can be used in the scientific research related to the blood glucose level.

Preferably, the SMO inhibitor includes any one or a combination of at least two of cyclopamine, vismodegib or glasdegib.

Preferably, the SMO inhibitor includes cyclopamine.

Compared with the prior art, the present disclosure has the following beneficial effects.

The present disclosure firstly studies the mechanism of vascular endothelial cell dysfunction caused by high glucose, and creatively finds that: 1. Hedgehog (Hh) signaling pathway of endothelial cells is highly activated under high glucose treatment, resulting in endothelial cell dysfunction; 2. high glucose induces the significant increase in the expression of SMO, STK36, and SHH in endothelial cells, where SMO, STK36, and SHH are the key genes in the Hh signaling pathway, and SMO is the most up-regulated gene; and 3. the targeting (knocking down) of the SMO gene can protect endothelial cells in the high glucose state and improve endothelial cell dysfunction (specifically, it can reduce LDH, reduce apoptosis, improve cell viability, promote cell proliferation, restore cell migration ability, and inhibit the expression of inflammatory cytokines TNF-α, MCP-1, and IL-6).

Based on the above mechanistic findings, the present disclosure further finds and confirms: 1. the SMO inhibitor can protect endothelial cells induced by high blood glucose and improve endothelial cell dysfunction (specifically, it reduces LDH, reduces apoptosis, and reduces the production of inflammatory cytokines TNF-α, MCP-1, and IL-6); 2. the SMO inhibitors can improve endothelial cell dysfunction by increasing the blood flow velocity, increasing inner diameter of the blood vessel, improving vascular intima thickening and reducing the production of inflammatory cytokines, thereby achieving the purpose of preventing, delaying or alleviating access stenosis of arteriovenous fistula; and 3. compared with other SMO inhibitors, cyclopamine (Cyc) has the best effect on protecting endothelial cells induced by high blood glucose and improving endothelial cell dysfunction and thus can prevent, delay or alleviate access stenosis of arteriovenous fistula more effectively.

The present disclosure successfully reduces the phenomenon of stenosis of AVF, protects the function of vascular endothelial cells, improves the patency rate of AVF and significantly prolongs the service life of the dialysis pathway through the intervention treatment of drugs (SMO inhibitors) in the AVF rat model of diabetic nephropathy.

The present disclosure starts with improving the stenosis of AVF in diabetic nephropathy, and with the use of the SMO inhibitor, achieves the purposes of preventing AVF stenosis caused by dysfunction of vascular endothelial cells in AVF for diabetic nephropathy, maintaining the blood flow, improving the patency rate of AVF and significantly prolonging the service life of the dialysis pathway, and the prevention or treatment scheme adopting the SMO inhibitor has the characteristics of safety, effectiveness and no side effects.

### BRIEF DESCRIPTION OF DRAWINGS

FIG. 1 is a graph showing the analysis results of transcriptomic profiling of human umbilical vein endothelial cells (HUVECs) in Example 1 under the stimulation of high glucose; wherein NG represents normal glucose and HG represents high glucose.

FIG. 2 is a graph showing the results of an in vitro HINEC high glucose stimulation experiment in Example 1 to verify the up-regulation of the SMO gene in the Hh signaling pathway; where A is the qRT-PCR analysis of SMO expression in HUVECs under different glucose concentrations; B is the western blot analysis of SMO expression in HUVECs under different glucose concentrations, wherein the upper graph of B is the representative graph of western blot, and the lower graph of B is the quantitative evaluation of western blot analysis results; Man represents the osmotic pressure control group and specifically 5.5 mM glucose+34.5 mM mannitol; 5.5 represents 5.5 mM glucose, that is, the normal glucose group; 11.1 represents 11.1 mM glucose; 25 represents 25 mM glucose; 40 represents 40 mM glucose, that is, the high glucose group; GAPDH is an internal reference protein in western blot.

FIG. 3 is a graph showing the results of SMO gene knockdown in Example 2 for increasing the viability of HUVEC cells, reducing apoptosis of HUVEC cells and promoting the proliferation of HUVEC cells under the stimulation of high glucose; where A is the results of cell viability; B is the results of apoptosis; C is the fluorescent results of cell proliferation (scale: 50 μm); D is the quantitative results of cell proliferation; shSMO represents SMO knockdown; shCtrl represents the control group, that is, the group free from SMO knockdown; HG represents high glucose treatment; Man is the osmotic pressure control group.

FIG. 4 is a graph showing the results of SMO gene knockdown in Example 2 for improving the ability of high glucose-induced HUVECs to release LDH, migrate and reducing the expression of inflammatory factors; where A is the determination of LDH release; B is the determination results of wound healing (scratch assay), the left graph represents the scratch migration graph at different time points (scale: 25 μm), and the right graph is the quantitative evaluation of the scratch migration analysis results; C is the results of the qRT-PCR analysis of the expression of inflammatory factors TNF-α, MCP-1, and IL-6; HG represents high glucose treatment; Man is the osmotic pressure control group; shSMO represents SMO knockdown; shCtrl represents the control group, that is, the group free from SMO knockdown.

FIG. 5 is a graph showing the effect of different SMO inhibitors in Example 3 on high glucose-treated HUVECs; where A is the results of the SMO expression level; B is the results of cell viability; C is the results of the IL-6 expression level; D is the results of the TNF-α expression level; E is the results of the MCP-1 expression level; HG represents high glucose treatment; NG represents normal glucose treatment; HG Cyc represents the intervention with Cyclopamine under high glucose treatment; HG T Vis represents the intervention with Vismodegib under high glucose treatment; HG Gla represents the intervention with Glasdegib under high glucose treatment.

FIG. 6 is a graph showing the results of the SMO inhibitor Cyc in Example 3 for improving the viability, LDH release and apoptosis of endothelial cells under the stimulation of high glucose; where A is the results of cell viability of HUVECs; B is the results of cell viability of PECs; C is the results of LDH release of HUVECs; D is the results of apoptosis of HUVECs; E is the results of apoptosis of PECs; PEC represents primary vascular endothelial cells (Primary EC); HG represents high glucose treatment; NG represents normal glucose treatment; Man represents the osmotic pressure control group; HG+Cyc represents the intervention with Cyclopamine under high glucose treatment.

FIG. 7 is a graph showing the results of Cyc in Example 3 for reducing the expression of high glucose-induced cell inflammatory factors; where A, B, and C are the results of the mRNA expression levels of IL-6, TNF-α, and MCP-1 in HUVECs, respectively; D is the representative graph of western blot of inflammatory factors; E, F, and G are the quantitative results of western blot of IL-6, TNF-α, and MCP-1, respectively; “−” represents no intervention with Cyc, and “+” represents the intervention with Cyc; 5.5 represents 5.5 mM glucose, that is, the normal glucose group; 11.1 represents 11.1 mM glucose; 25 represents 25 mM glucose; 40 represents 40 mM glucose; GAPDH is an internal reference protein in western blot.

FIG. 8 is a graph showing the results of the SMO inhibitor Cyc in Example 3 for reducing the secretion level of inflammatory factors in cells under the stimulation of high glucose; where A, B, and C are the results of the secretion levels of IL-6, TNF-α, and MCP-1 in HUVECs, respectively; “−” represents no intervention with Cyc, and “+” represents the intervention with Cyc; 5.5 represents 5.5 mM glucose, that is, the normal glucose group; 11.1 represents 11.1 mM glucose; 25 represents 25 mM glucose; 40 represents 40 mM glucose.

FIG. 9 is a graph showing the effect results of Cyc on the body weight of diabetic AVF rats in Example 4.

FIG. 10 is a graph showing the effect results of Cyc on the blood glucose level of diabetic AVF rats in Example 4.

FIG. 11 is a graph showing the results of in vitro ultrasound examination of the AVF of rats in Example 4; where A is the ultrasound of blood flows near the AVF anastomotic stoma at 0 s, 15 s and 30 s, B is the diameter of blood vessels near the AVF anastomotic stoma under ultrasound, and C is the peak flow velocity near the AVF anastomotic stoma under ultrasound.

FIG. 12 is a graph showing the results of hematoxylin-eosin staining changes of the AVF anastomotic vessels of rats in Example 4, at a scale of 50 μm.

FIG. 13 is a graph showing immunohistochemical characteristics of SMO, IL-6, TNF-α, MCP-1, and cleaved caspase-3 in the AVF tissue of rats in Example 4, at a scale of 50 μm.

FIG. 14 is a graph showing the results of Cyc in Example 4 for reducing the expression of inflammatory factors in diabetic AVF rats; where A, B, and C are the levels of TNF-α, MCP-1, and IL-6 in plasma, respectively, and 0, 1 and 14 weeks represent the normal stage, the diabetic stage and the diabetic AVF stage, respectively; D is the representative graph of western blot of SMO, TNF-α, MCP-1, and IL-6 in vascular AVF tissue of rats, respectively; E, F, G, and H are the quantitative evaluation of western blot results of IL-6, TNF-α, MCP-1, and SMO, respectively.

### DETAILED DESCRIPTION

The technical solutions of the present disclosure will be further described through examples below. It is apparent to those skilled in the art that the examples are intended to aid in understanding the present disclosure and should not be taken as a specific limitation of the present disclosure.

In the following examples, unless otherwise specified, all reagents and consumables are purchased from conventional reagent manufacturers in the art; unless otherwise specified, the experimental methods and technical means used are conventional methods and means in the art.

### DEFINITION OF TERMS

The term “arteriovenous fistula (AVF)” as used in the present disclosure refers to a minor operation for vascular anastomosis, in which the artery of the forearm near the wrist is sutured with the adjacent vein so that arterial blood is allowed to flow in the anastomosed vein to form an arteriovenous fistula, and is mainly used for hemodialysis treatment, providing sufficient blood for hemodialysis treatment and guaranteeing the adequacy of dialysis treatment.

The term “endothelial cell dysfunction” as used in the present disclosure refers to various non-adaptive changes in the function of endothelial cells and has important effects on hemostasis, local angiotasis, redox balance, and acute and chronic inflammatory responses.

The term “diabetic nephropathy AVF rat” used in the present disclosure refers to that after the SD rat is constructed as a diabetic model, the iliac artery and iliac vein are anastomosed into a fistula to form an AVF model by surgical intervention. For the construction method, reference is made to Example 4.

The experimental methods involved in the following examples are as follows.

RNA Sequencing (RNA-Seq)

HUVECs were inoculated in a 6-well plate (4×10³-1×10⁵) and cultured in different glucose treatment conditions for 48 hours, and the cells of each treatment groups were collected. Cells were washed with PBS, and TRlzol was added. RNA extraction, sequencing library construction, and sequencing were carried out by Shanghai NovelBio Technology Co., Ltd. Sequencing was carried out using the Illumina NovaSeq 6000 platform. Differentially expressed genes (DEGs) were identified using the DESeq package. The identification standard of differentially expressed genes was that P was less than 0.01 and the fold change was greater than 1.5.

Fluorescence Quantitative PCR (qPCR)

The total RNA was extracted from HUVECs by the Trizol method. The cDNA was synthesized using Evo M-MLV RT Premix premix for qPCR. Fluorescence quantitative PCR was carried out using gene-specific primers (Table 1) and SYBR® Green Premix Pro Taq HS qPCR Kit. The relative mRNA expression quantity was calculated by 2^{-ΔΔCT} method.

TABLE 1
Primer sequence of quantitative real-time PCR

| Gene | Primer sequence (5′→3′) | | Serial number |
|---|---|---|---|
| GAPDH | F: | GACCTGCCGTCTAGAAAAAC | SEQ ID NO. 1 |
| | R: | CTGTAGCCAAATTCGTTGTC | SEQ ID NO. 2 |
| SMO | F: | ACCTATGCCTGGCACACTTC | SEQ ID NO. 3 |
| | R: | AGGAAGTAGCCTCCCACGAT | SEQ ID NO. 4 |
| TNF-α | F: | TGGGATCATTGCCCTGTGAG | SEQ ID NO. 5 |
| | R: | GGTGTCTGAAGGAGGGGGTA | SEQ ID NO. 6 |
| MCP-1 | F: | CCCCAGTCACCTGCTGTTAT | SEQ ID NO. 7 |
| | R: | AGATCTCCTTGGCCACAATG | SEQ ID NO. 8 |
| IL-6 | F: | TTTTGGTGTTGTGCAAGGGTC | SEQ ID NO. 9 |
| | R: | ATCGCTCCCTCTCCCTGTAA | SEQ ID NO. 10 |

Apoptosis Detection

HUVECs were inoculated into a 6-well plate and treated with different concentrations of glucose. After 48 hours of culture, the cells were collected, washed twice with cold PBS, and suspended to 1×10⁶ cells/mL with a labeling buffer (1×), and 5 μL of AnnexinV-FITC and 5 μL of propidium iodide (PI) were added. The cells were mixed evenly and incubated at room temperature away from light for about 15 minutes. 300 μL of labeling buffer was added to each tube, and samples were loaded within 1 hour. Annexin-V and PI were used to label early and late apoptotic cells, respectively. Annexin-V is a Ca²⁺-dependent phospholipid-binding protein with a molecular weight of 35-36 KD, can bind specifically to phosphatidylserine with high affinity, and is a sensitive index for detecting early apoptotic cells (early apoptotic cells are positive). PI is a kind of nucleic acid dye, cannot penetrate the whole cell membrane, but can penetrate the cell membrane of cells in the middle and late apoptosis and dead cells to stain the nucleus.

CCK8 Experiment

1×10⁴ HUVECs were inoculated in each well of a 96-well plate, and glucose of different concentrations was added for grouping treatment, where 5.5 mM glucose was used as the normal glucose concentration, and Man was used as the osmotic pressure control. After 48 hours of culture, 10 μL of CCK8 solution was added to each well, and the cells were incubated in an incubator for 0.5-4 hours. The experimental principle is as follows: 2-(2-methoxy-4-nitrophenyl)-3-(4-nitrophenyl)-5-(2,4-disulfophenyl)-2H-tetrazolium sodium salt was reduced by dehydrogenase in cells to yellow Formazan dye with high water solubility under the action of the electron carrier 1-methoxy-5-methylphenazinium methyl sulfate (1-Methoxy PMS). The amount of produced Formazan dye was directly proportional to the number of living cells. Finally, the light absorption value at 450 nm wavelength was detected by a microplate reader. In a certain range, the number of living cells was directly proportional to the absorption value. In the experiment, each experimental group had more than 3 duplications, and the average value was taken.

EdU Detection of Cell Proliferation

HUVECs were inoculated in a 96-well plate (4×10³-1×10⁵ cells per well) and cultured in normal glucose (5.5 mM glucose) and high glucose (40 mM glucose) for 48 hours. The newly synthesized DNA of cells were detected by EdU according to the instructions of the cell proliferation EdU kit, double labeling was carried out in combination with a nuclear marker (DAPI) to detect cell proliferation, and the results were observed with a fluorescence microscope.

Enzyme-Linked Immunosorbent Assay (ELISA)-Cell

HUVECs were inoculated into a 6-well plate and treated with glucose of different concentrations. After 48 hours, the supernatant was collected, and the expression of inflammatory cytokines TNF-α, MCP-1. and IL-6 in the supernatant was detected using TNF-α, MCP-1 and IL-6 ELISA kits according to instructions of the kits, respectively.

Lactate Dehydrogenase (LDH) Assay

HUVECs were inoculated in a 6-well plate (1×10⁶ cells/well) and treated in different glucose conditions. After 48 hours of culture, the supernatant was collected, and the LDH activity was analyzed using an LDH cytotoxicity assay kit.

SMO Gene Knockdown (Plasmid Construction, Lentivirus Production and Lentivirus Transduction)

A stable knockdown cell line was produced using a lentivirus system. SMO shRNA vector was purchased from Guangzhou VectorBuilder Inc. All constructs were confirmed by Sanger sequencing, and the shRNA targeting sequences used in this study are detailed in Table 2. Lentivirus production: Lentivirus plasmids were transfected into 293T cells. Then, the lentivirus supernatant was collected and filtered with a 0.22 μM filter after 48 hours of transfection. HUVECs were infected with lentivirus. After 48 hours of infection, puromycin (2 μg/mL) was added to the culture medium, and positive infected cells were selected.

TABLE 2
SMO shRNA targeting sequence (5′→3′)

| Vector | Targeting sequence |
|---|---|
| bSMO [shRNA#1] | ATCGCTACCCTGCTGTTATTC |
| | (SEQ ID NO. 11) |
| bSMO [shRNA#2] | ACGTCAATGCGTGCTTCTTTG |
| | (SEQ ID NO. 12) |
| bSMO [shRNA#3] | TCAACCTGTTTGCCATGTTTG |
| | (SEQ ID NO. 13) |

Scratch Experiment

HUVECs were inoculated in a 6-well plate and incubated in the high glucose (40 mM glucose) conditions for 48 hours. The cells were inoculated in a 6-well plate, starved in a serum-free medium for 4-6 hours after the cells grew to cover the bottom of the covering plate, and streaked in a parallel fashion using a tip of a 100 μL sterile micropipette with the tip vertical without tilt. The old culture medium was removed, the plate were washed with sterile PBS with the scratched cells removed, and the corresponding culture medium was added. Cell migration was observed with an inverted microscope at 0, 6, 12 and 24 hours, respectively, and analyzed using Image-Pro Plus 6.0 software.

Ultrasonic Examination

The hemodynamic changes of iliac vessels were examined using an ultra-high frequency high resolution small animal ultrasonic imaging system of a 13-24 Mhz linear transducer. Rats were anesthetized with chloral hydrate (0.3 mL/100 g) and placed in the supine position. Hair in the groin area was removed using a shaving apparatus to minimize ultrasonic attenuation. Under continuous anesthesia, the rats were examined by ultrasound by experienced experimenters. The peak blood flow velocity and vessel diameter of the proximal iliac vein (the venous side of AVF) of the rats were measured by gray-scale and Doppler ultrasound. When the peak blood flow velocity was measured, the Doppler sample size was adjusted to 0.5 mm, and the irradiation angle was kept constant less than 60° according to the diameter and direction of blood vessels. The diameter of blood vessels was measured near the anastomotic stoma of iliac vascular fistula. All observed data were measured three times.

Hematoxylin-Eosin (HE) and Immunohistochemical (IHC) Staining

The paraffin-embedded tissue sections were stained with HE. Blood vessel tissue samples were dewaxed and rehydrated, and antigen retrieval and IHC staining were carried out. Tissue sections were blocked at room temperature for at least 1 hour and incubated overnight with antibodies of TNF-α, MCP1, IL-6, and SMO at 4° C. After careful cleaning, tissue sections were detected using a DAB-HRP method and observed with a microscope. Quantitative analysis was carried out according to the percentage of living cells and the staining intensity.

Enzyme-Linked Immunosorbent Assay (ELISA)-Plasma

Peripheral blood samples of rats were collected, and plasma was separated. The concentration of cytokines (TNF-α, MCP-1, and IL-6) in plasma was determined using an ELISA kit (Jiangsu Enzyme Immune Industrial Co., Ltd., China).

Western Blot

After the experiment was finished, the AVF tissues of the rats were collected and digested with enzymes to separate vascular endothelial cells. The vascular endothelial cells were lysed with the mixture liquid of RIPA lysis buffer and the phosphatase inhibitor and centrifuged to collect the supernatant. The protein concentration was measured, and the protein samples were prepared. Electrophoresis was carried out, and the membrane was transferred at 260 mA for 60 minutes. The transferred PVDF membranes were soaked with TBST buffer containing 5% skimmed milk powder, blocked at room temperature at a rotation rate of 60 rpm for 1 hour, and gently washed with TBST buffer 3 times, each time lasting 8-10 minutes. The corresponding diluents of primary antibodies (SMO, Cascpase3, TNF-α, MCP-1, and IL-6) were added, where the dilution ratio was determined according to the requirements of each antibody, and the PVDF membranes were incubated overnight in a shaker at 4° C. and cleaned with TBST buffer 3 times the next day, each time lasting 8-10 minutes. The diluents of secondary antibodies of the corresponding anti-species sources were added to the PVDF membranes, and the PVDF membranes were incubated at room temperature for 1 hour and gently washed with the TBST buffer 3 times, each time lasting 8-10 minutes. Development: Luminescent liquid A and liquid B were mixed according to the ratio of 1:1, the residual water on the PVDF membranes was gently absorbed with filter papers, the mixed luminescent liquid was uniformly dropped on the PVDF membranes, and the western blot results were analyzed on a gel imager.

### Example 1

Detection of the Mechanism of Vascular Endothelial Cell Dysfunction Induced by High Glucose at the Cellular Level

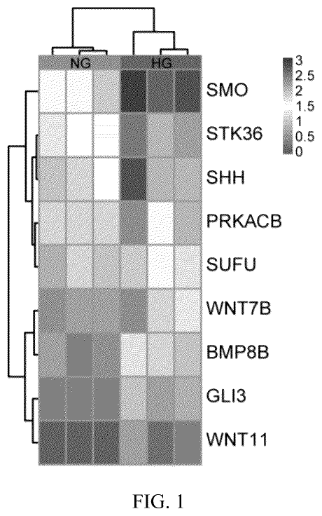

The present disclosure firstly studied the mechanism that high glucose (HG) stimulated the production and release of cytokines and impairs the function of endothelial cells at the cellular level. In the present disclosure, HUVECs were treated with 40 mM glucose for 48 hours, and RNA sequencing (RNA-seq) analysis was carried out on the HUVECs. The results showed that after the HG treatment, the gene expression of most metabolic genes and signaling pathways changed widely. It is to be noted that HG induced a significant increase in the expression levels of SMO, STK36, and SHH, where SMO, STK36, and SHH were key genes in the Hh signaling pathway. Consistent with the preceding results, the KEGG pathway enrichment analysis also confirmed that compared with the control group (normal glucose, NG), the Hh signaling pathway was highly activated in HUVECs treated with high glucose. Among Hh signaling pathway genes, SMO was the most up-regulated gene in the HUVECs treated with high glucose (FIG. 1).

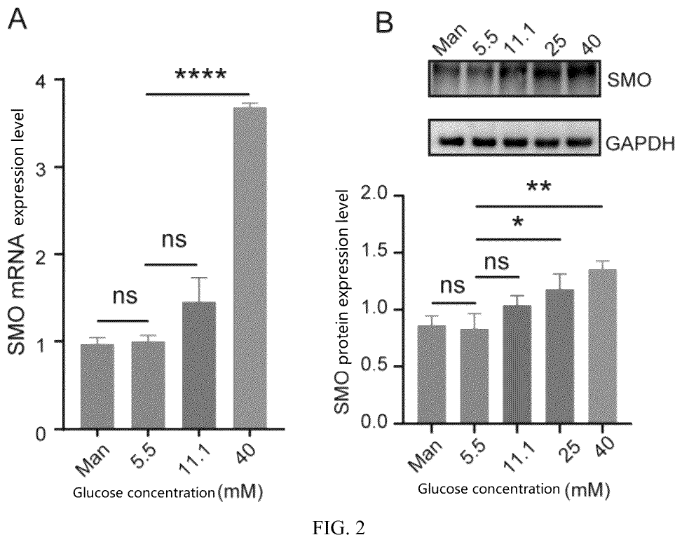

In order to verify the preceding RNA-seq results, the present disclosure detected the mRNA expression of SMO in HUVECs subjected to different treatments. Consistent with the preceding results, the high glucose treatment strongly up-regulated the mRNA expression of SMO (FIG. 2A). Western blot analysis also confirmed the up-regulation of SMO at the protein level under the stimulation of HG (FIG. 2B). In conclusion, the results of Example 1 showed that: 1. the Hh signaling pathway of HUVECs was highly activated under high glucose treatment; 2. high glucose induced a significant increase in the expression of SMO, STK36, and SHH in HUVECs, where SMO, STK36, and SHH were the key genes in the Hh signaling pathway, and SMO was the most up-regulated gene.

### Example 2

Hh Signaling Pathway Leads to Endothelial Dysfunction

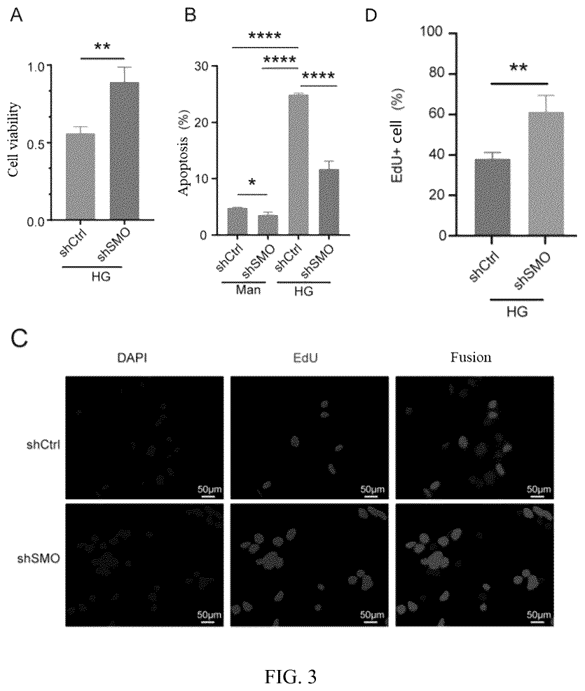

On the basis of Example 1, the present disclosure continued to study whether the Hh signaling pathway leads to endothelial dysfunction. SMO knockdown cell lines were constructed first, and their cytokine production, cell viability, proliferation and apoptosis were examined in the high glucose conditions. The results showed that in the high glucose conditions, compared with the control group (cell lines free from SMO knockdown), SMO knockout significantly increased the activity of HUVECs (FIG. 3A), reduced the apoptosis of endothelial cells (FIG. 3B), and promoted the proliferation of endothelial cells (FIG. 3C). These results indicated that SMO knockdown could protect endothelial cells from dysfunction under high glucose.

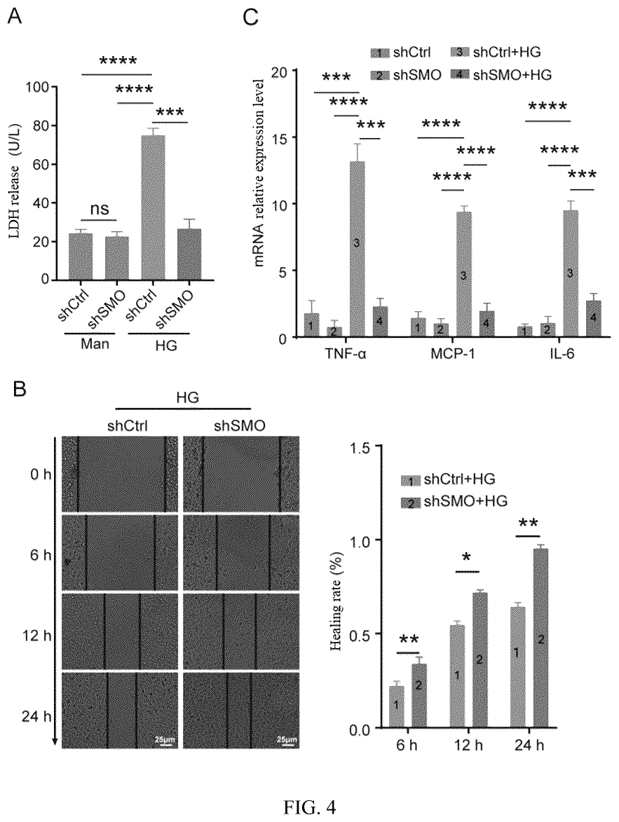

In order to further determine whether knocking down SMO can restore endothelial cell dysfunction, the present disclosure detected the LDH level of HUVECs with SMO knocked down under high glucose treatment. As expected, results showed that compared with the control group, the LDH of HUVECs decreased significantly after SMO knockdown (FIG. 4A); it is to be noted that the migration ability of HUVECs had also been significantly restored after SMO knockdown (FIG. 4B); moreover, knocking down SMO could significantly inhibit the expression of inflammatory cytokines TNF-α, MCP-1, and IL-6 in HUVECs (FIG. 4C). In general, these results fully showed that the Hh signaling pathway led to the endothelial cell dysfunction and targeting SMO could protect endothelial cells in the high glucose state and improve their dysfunction.

In conclusion, the results of Example 2 showed: 1. the Hh signaling pathway of HUVECs was highly activated under high glucose treatment, resulting in endothelial cell dysfunction; and 2. the targeting (knocking down) of the SMO gene could protect endothelial cells in the high glucose state and improve endothelial cell dysfunction (specifically, it reduced LDH, reduced apoptosis, improved cell viability, promoted cell proliferation, restored cell migration ability, and inhibited the expression of inflammatory cytokines TNF-α, MCP-1, and IL-6).

### Example 3

Screening and Functional Validation of SMO Inhibitors in the Hh Signaling Pathway

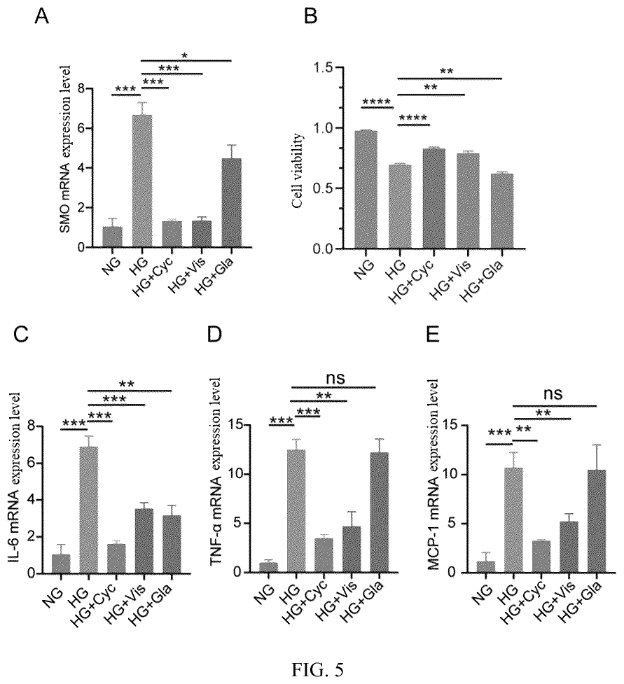

The present disclosure screened several SMO inhibitors, including Cyc (Cyclopamine, purchased from Guangzhou Zuoke Biotechnology Development Co., Ltd., MedChemExpress, HY-17024), Vis (Vismodegib, purchased from Guangzhou Zuoke Biotechnology Development Co., Ltd., MedChemExpress, HY-10440), and Gla (Glasdegib, purchased from Guangzhou Zuoke Biotechnology Development Co., Ltd., MedChemExpress, HY-16391). The results showed that at the same dosage (10 μM), Cyc had the best effect on inhibiting the expression of SMO, increasing the cell viability of HUVECs treated with high glucose, and inhibiting the expression of inflammatory factors TNF-α, MCP-1, and IL-6 of HUVECs treated with high glucose (FIG. 5).

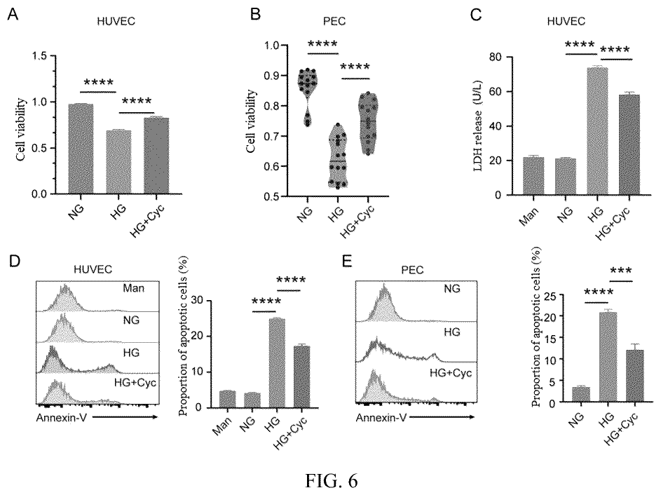

In order to test whether the SMO inhibitor can reproduce the effect of the knockdown of SMO, the present disclosure firstly verified the cell vitality recovery of endothelial cells in the presence of Cyc (10 μM) in HG culture conditions. As expected, the intervention with Cyc could significantly restore the cell viability of HUVECs in the presence of HG (FIG. 6A). Similar results were obtained in Primary ECs (PECs) cultured in HG conditions in vitro (FIG. 6B). In addition, the present disclosure also detected the expression level of LDH in HUVECs after the intervention with Cyc and found that LDH could be reduced after the intervention with Cyc (FIG. 6C). Consistent with the preceding results, the addition of Cyc significantly reduced the apoptosis of HUVECs and cultured PECs in high glucose conditions (FIGS. 6D-6E).

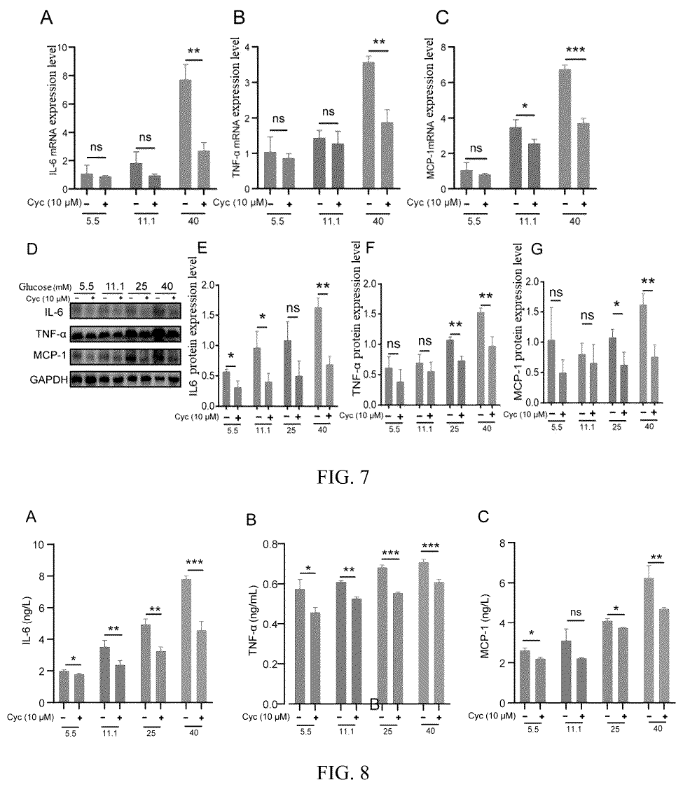

Then, whether Cyc can reduce the excessive production of inflammatory cytokines was tested. The results showed that Cyc could inhibit the expression of IL-6, MCP-1, and TNF-α at mRNA and protein levels (FIGS. 7A-7G).

In addition, the levels of TNF-α, MCP-1, and IL-6 were detected to be decreased in the Cyc group by ELISA (the relative content of inflammatory factors in the cell culture supernatant was detected after HUVECs were treated with 10 μM of Cyc) (FIGS. 8A-8C). These results indicated that the SMO inhibitors could save the function of HUVECs and PECs in vitro and reduce the production of inflammatory cytokines. These data indicated that the SMO inhibition could be used as a method to improve endothelial dysfunction induced by hyperglycemia.

In conclusion, the results of Example 3 showed: 1. the SMO inhibitor could protect endothelial cells induced by high blood glucose and improve endothelial cell dysfunction (specifically, it could reduce the expression of LDH, reduce apoptosis, and reduce the production of inflammatory cytokines TNF-α, MCP-1, and IL-6); and 2. compared with other SMO inhibitors, Cyc had the best effect on protecting endothelial cells induced by high blood glucose and improving endothelial cell dysfunction.

### Example 4

Therapeutic Effect of Cyc on Diabetic AVF Rats

(1) Construction of the Diabetic Model in Rats

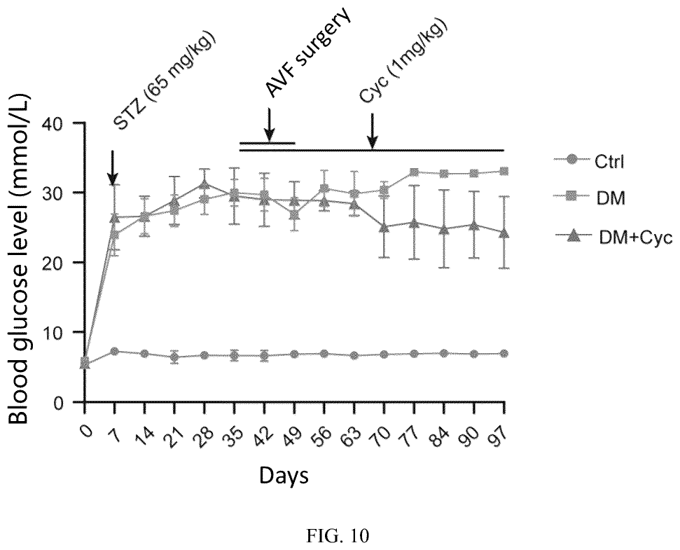

Male SD rats which were aged 6-8 weeks and weighed 200-250 g were purchased from Beijing Vital River Laboratory Animal Technology Co., Ltd. In order to induce diabetes, the rats fasted overnight. Streptozotocin (STZ) (US, sigma, S0130-1G) (65 mg/kg) was dissolved in the citric acid buffer solution (0.1 M citric acid and 0.1 M sodium citrate) and single injected intraperitoneally into the abdominal cavity of the rats. The control group was administrated with an equal volume of the mediator-sodium citrate buffer (pH 4.5). Three days after the STZ injection, SD rats whose fasting-blood glucose (FPG) was greater than or equal to 300 mg/dL measured by Roche glucometer (Roche, Shanghai, China, LKBIO1500) were considered to have diabetes and used in follow-up experiments (FIG. 10).

(2) Construction of the AVF Model in Rats (AVF Surgery)

After the construction of the diabetic model. SD rats were used to construct the AVF model. Rats were anesthetized by intraperitoneal injection of chloral hydrate (0.3 mL/100 g) and placed on the operating table during the operation. The iliac vascular area was exposed by cutting a 3 cm incision along the left inguinal fold and retracting abdominal muscle tissue and other soft tissue. The operation was carried out under a microscope, and the iliac artery and vein were separated from the surrounding fascia and nerves. Then, the vein was ligated at the exposed distal end, the non-invasive clip was clamped at the exposed proximal end, and the ligated proximal end was cut at an angle of 45°. At the site where the anastomotic stoma was formed, a small longitudinal incision was made in the vein with a miniature scalpel. The lumens of the two vessels were flushed with heparin saline, and the vein and the artery were intermittently sutured end to side with a 9-0 monofilament nylon suture. The AVF flow was confirmed by the naked eyes through bright red arterial blood at anastomotic stoma. A weak pulse could be felt at the proximal end of iliac vein.

(3) The Rats were Divided into a Ctrl Group, a DM Group, and a DM+Cyc Group, with Four Rats in Each Group:

TABLE 3

| | | | Day 35 to end |
|---|---|---|---|
| | | | (The experiment lasted 14 |
| Group | Day 7 | Day 35 | weeks) |
| Ctrl | \ | AVF surgery | \ |
| DM | 65 mg/kg STZ | AVF surgery | \ |
| DM + Cyc | 65 mg/kg STZ | AVF surgery | Intraperitoneal injection of |
| | | | Cyc 1 mg/kg/day (solvent: |
| | | | PBS) |

Weight and blood glucose of rats were measured every week during the experiment, peripheral blood was extracted at specific time points (0, 1, and 14 weeks) for analysis, and AVF tissues were isolated at the end of the experiment for further analysis.

(4) Effect of Cyc on Body Weight and Blood Glucose Level of Diabetic AVF Rats

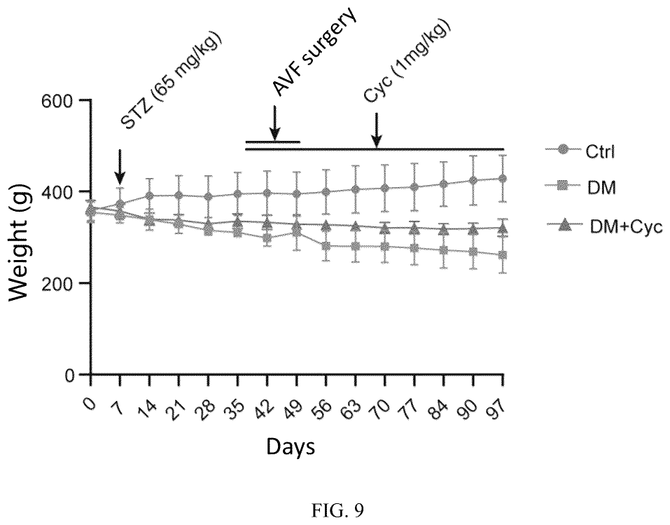

The monitoring results of the body weight showed (FIG. 9) that the weight of rats without diabetics (Ctrl group) gradually increased, while the weight of diabetic rats (DM group) gradually decreased. Compared with the DM group, the body weight of diabetic rats treated with the SMO inhibitor Cyc (DM+Cyc group) remained stable over time.

The monitoring results of the blood glucose level showed (FIG. 10) that although the blood glucose level of diabetic rats treated with Cyc (DM+Cyc group) was lower than that of the DM group, the blood glucose level was still quite different from that of rats without diabetes (Ctrl group), which indicated that the improvement of endothelial cell dysfunction by Cyc was not achieved through the reduction of the blood glucose level.

(5) Cyc Improves the Peak Flow Velocity and Inner Diameter of Blood Vessels

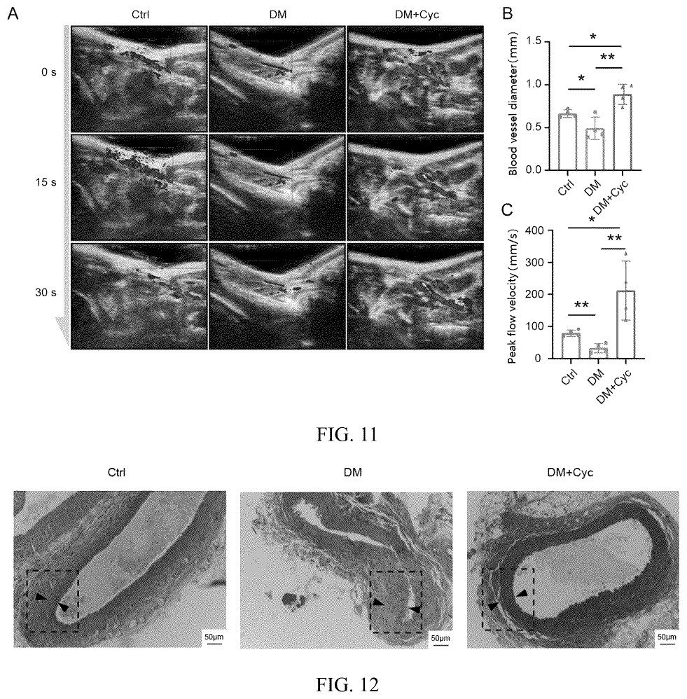

In order to evaluate whether the SMO inhibitor could prevent or delay fistula stenosis, the present disclosure carried out the color Doppler ultrasonic examination on vessels of rats in the above groups to detect changes in AVF of the hemodialysis vascular access (FIG. 11A). Compared with the untreated DM group, the blood flow of the DM+Cyc group treated with Cyc was faster. The present disclosure also measured the diameter of blood vessels at AVF anastomosis by ultrasound. The data of the present disclosure showed that compared with rats without diabetics (Ctrl group), the diameter of blood vessels of untreated diabetic rats (DM group) was significantly reduced, which indicated that the hemodialysis vascular access was narrowed. Interestingly, compared with the untreated group, the diameter of blood vessels in the fistula of rats treated with Cyc (DM+Cyc group) increased significantly, even higher than that of rats without diabetes (Ctrl group) (FIG. 11B), and a similar trend was observed in the peak flow velocity (FIG. 11C), which indicated that Cyc effectively prevented the occurrence of AVF stenosis and could delay or prevent the occurrence of AVF stenosis by improving the peak flow velocity and inner diameter of blood vessels.

(6) Cyc Improves the Vascular Intima Thickening of Diabetic AVF and Reduces the Production of Inflammatory Cytokines

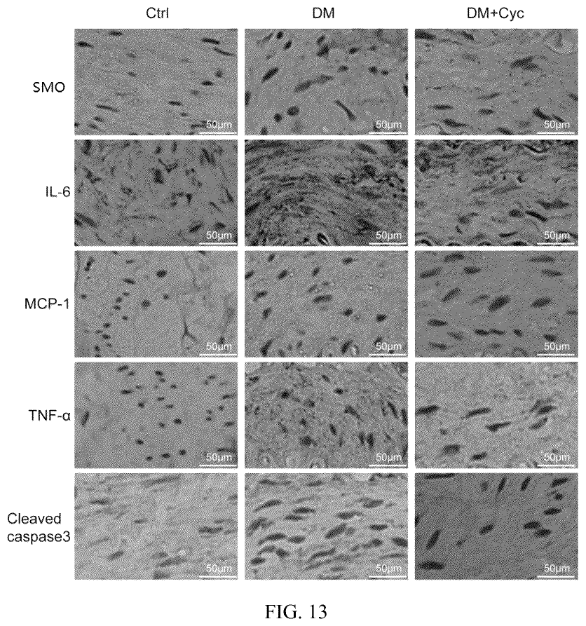

In order to understand the influence of high glucose on the development of pathological changes, in the present disclosure, the vascular tissue at AVF anastomosis was stained with hematoxylin-eosin. Compared with the rats without diabetics (Ctrl group), the intima of diabetic rats (DM group) was significantly thickened, and the lumen was relatively narrow. Surprisingly, after the intervention with the SMO inhibitor Cyc, the intima of blood vessels was improved, and the lumen of blood vessels was significantly enlarged (FIG. 12). In order to further study whether the effect of Cyc on stenosis prevention was related to the SMO inhibition and the reduction of endothelial cell dysfunction, the present disclosure carried out immunohistochemical analysis on vascular tissue around the fistula. The results (FIG. 13) showed that compared with rats in the group without diabetics (Ctrl group), the expression levels of SMO, TNF-α, MCP-1, IL-6, and Cleaved caspase 3 of rats in the diabetic group (DM group) increased, and the expression levels of these proteins were significantly inhibited after the intervention with Cyc, which indicated that the intervention with Cyc reduced the production of inflammatory cytokines.

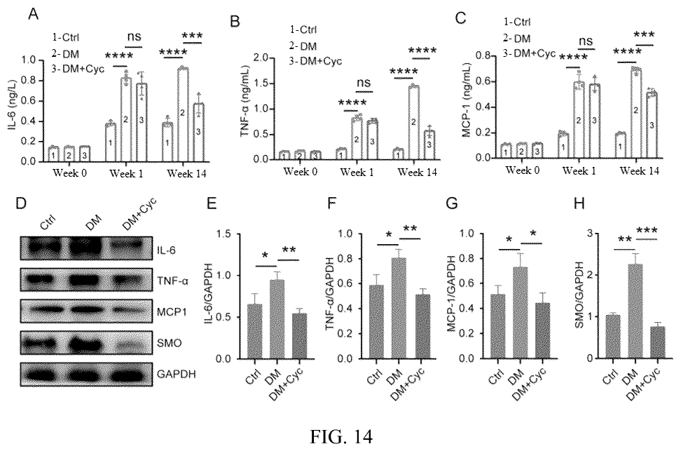

The present disclosure further measured the levels of inflammatory factors in the peripheral blood plasma of rats in each group by ELISA (FIGS. 14A-14C), where the 0, 1 and 14 weeks respectively represented the normal stage, the diabetic stage and the diabetic AVF stage. As expected, the present disclosure found that compared with rats without diabetics (Ctrl group), TNF-α, MCP-1, and IL-6 significantly increased in plasma of diabetic rats (DM group). After the intervention of Cyc, these inflammatory factors were significantly reduced. In addition, the present disclosure carried out a western blot experiment on vascular endothelial cells isolated from rat vascular AVF tissue and also obtained the result that the intervention with Cyc significantly reduced the expression levels of SMO, TNF-α, MCP-1, and IL-6 (FIGS. 14D-14H). These results indicated that the therapeutic inhibition of SMO could prevent stenosis, probably due to the decrease of inflammatory cytokines produced by endothelial cells in high blood glucose caused by diabetes.

In conclusion, the results of Example 4 showed: the SMO inhibitor Cyc could improve endothelial cell dysfunction by increasing the peak blood flow velocity in blood vessels, increasing the inner diameter of the blood vessel, improving the vascular intima thickening and reducing the production of inflammatory cytokines, thereby achieving the purpose of preventing, delaying or alleviating the access stenosis of arteriovenous fistula.

The applicant claims that the present disclosure illustrates the use of the SMO inhibitor of the present disclosure in the preparation of a drug for preventing, delaying or alleviating access stenosis of arteriovenous fistula through the preceding examples, but the present disclosure is not limited to the preceding examples, that is, it means that the implementation of the present disclosure does not necessarily depend on the preceding examples. It is apparent to those skilled in the art that any modification of the present disclosure, equivalent substitution of each raw material of the product of the present disclosure, addition of auxiliary components, and selection of specific methods are within the scope of protection and disclosure of the present disclosure.

Preferred embodiments of the present disclosure have been described in detail above, but the present disclosure is not limited to the specific details in the preceding embodiments. Within the scope of the technical conception of the present disclosure, various simple modifications can be made to the technical solutions of the present disclosure, and these simple modifications all belong to the protection scope of the present disclosure.

In addition, it is to be noted that the specific technical features described in the preceding embodiments can be combined in any suitable manner without contradiction, and various possible combination modes are not further described in the present disclosure in order to avoid unnecessary repetition.

What is claimed is:

1. A method for preventing, delaying or alleviating access stenosis of arteriovenous fistula, comprising administering an effective amount of a smoothened (SMO) inhibitor to subject in need thereof.

2. The method of claim 1, wherein the SMO inhibitor comprises any one or a combination of at least two of cyclopamine, vismodegib or glasdegib.

3. The method of claim 1, wherein the SMO inhibitor comprises cyclopamine.

4. The method of claim 1, wherein a dosage form of the drug comprises a solution, a tablet, a capsule or a granule.

5. The method of claim 1, wherein the drug comprises a pharmaceutically acceptable adjuvant.

6. The method of claim 5, wherein the adjuvant comprises any one or a combination of at least two of a diluent, a disintegrant, a flavoring agent, an adhesive, an excipient or a filler.

7. A method for preventing or treating vasculitis caused by endothelial injury, comprising administering an effective amount of a smoothened (SMO) inhibitor to subject in need thereof.

8. The method of claim 7, wherein the SMO inhibitor comprises any one or a combination of at least two of cyclopamine, vismodegib or glasdegib.

9. The method of claim 7, wherein the SMO inhibitor comprises cyclopamine.

10. A method for increasing the inner diameter of the blood vessel, comprising administering an effective amount of a smoothened (SMO) inhibitor to subject in need thereof.

11. The method of claim 10, wherein the SMO inhibitor comprises any one or a combination of at least two of cyclopamine, vismodegib or glasdegib.

12. The method of claim 10, wherein the SMO inhibitor comprises cyclopamine.
